(19) United States
(12) Patent Application Publication
(10) Pub. No.: US 2014/0107601 A1
(43) Pub. Date: Apr. 17, 2014

(54) Catheter Bag Holder and Method of Use

(71) Applicants: Mike BRANCH, Magnolia, TX (US); Patricia BRANCH, Magnolia, TX (US)

(72) Inventors: Mike BRANCH, Magnolia, TX (US); Patricia BRANCH, Magnolia, TX (US)

(21) Appl. No.: 14/056,920

(22) Filed: Oct. 17, 2013

Related U.S. Application Data
(60) Provisional application No. 61/715,281, filed on Oct. 17, 2012.

(51) Int. Cl. A61F 5/44

(52) U.S. Cl. CPC A61F 5/4408
USPC 604/327

(57) ABSTRACT

A catheter bag holder comprising a pocket having a top opening, a bottom opening, a pocket width and a pocket height. A one or more straps comprising a first strap and a second strap. The pocket having a top portion and a bottom portion. The top opening is in the top portion and the bottom opening is in the bottom portion. The one or more straps comprise an elastic material. The top opening having a top opening width and the bottom opening having a bottom opening width. The pocket width is greater than or equal to a bag width of a fluid bag. The bottom opening width of the pocket is smaller than the bag width. The first strap is attached to the top portion of the pocket. The second strap is attached to the bottom portion of the pocket. The pocket receiving a input tube through the top opening.

### BACKGROUND

This nonprovisional application claims the benefit of the provisional U.S. Patent Application No. 61/715,281, filed on Oct. 17, 2012.

This disclosure relates generally to a catheter bag holder. One example of another catheter bag holder can be found in U.S. Design Pat. No. D424,196. However, none of the known inventions and/or patents, taken either singularly or in combination, is seen to describe the instant disclosure as claimed. Accordingly, an improved catheter bag holder would be advantageous.

### SUMMARY

A catheter bag holder and a method of using a catheter bag holder are disclosed.

Said catheter bag holder comprising a catheter bag holder comprising a pocket having a top opening, a bottom opening, a pocket width and a pocket height. A one or more straps comprising a first strap and a second strap. Said pocket having a top portion and a bottom portion. Said top opening is in said top portion and said bottom opening is in said bottom portion. Said one or more straps comprise an elastic material. Said top opening having a top opening width and said bottom opening having a bottom opening width. Said pocket width is greater than or equal to a bag width of a fluid bag. Said bottom opening width of said pocket is smaller than said bag width. Said first strap is attached to said top portion of said pocket. Said second strap is attached to said bottom portion of said pocket. Said pocket receiving a input tube through said top opening.

A method of using a catheter bag holder comprising: attaching a catheter bag holder to a first leg of a user of said catheter bag holder by wrapping a one or more straps around a thigh of said first leg of said user, attaching said one or more straps to a pocket of said catheter bag holder, attaching a catheter to a groin area of said user for collection of bodily fluids from said user, attaching said catheter to a input tube of a fluid bag, inserting said fluid bag into said pocket of said catheter bag holder through a top opening of said catheter bag holder, and running a output tube from said fluid bag through a bottom opening of said pocket. Said catheter bag holder comprises said pocket having a pocket width and a pocket height. Said one or more straps comprising a first strap and a second strap. Said pocket comprises a top portion and a bottom portion. Said top opening is in said top portion and said bottom opening is in said bottom portion. Said one or more straps comprise an elastic material. Said top opening having a top opening width and said bottom opening having a bottom opening width. Said pocket width is greater than or equal to a bag width of said fluid bag. Said bottom opening width of said pocket is smaller than said bag width. Said first strap is attached to said top portion of said pocket. Said second strap is attached to said bottom portion of said pocket. Said pocket receiving a input tube through said top opening.

### BRIEF DESCRIPTION OF THE DRAWINGS

FIG. 1 illustrates a perspective overview of a catheter bag holder on a user.

FIGS. 2A, 2B, 2C and 2D illustrate a perspective front and back view, an elevated side view and an elevated front view of said catheter bag holder.

FIG. 3 illustrates a perspective overview of one embodiment of said one or more straps and said one or more sleeves.

FIGS. 4A, 4B, 4C and 4D illustrate four elevated views of said pocket with said one or more sleeves; including a top view, a front view, a side view and bottom view.

FIGS. 5A and 5B illustrate an elevated front view and an elevated rear view of said catheter bag holder.

FIG. 5C illustrates said catheter bag holder with a one or more buckles.

FIG. 6 illustrates an elevated front view of said catheter system.

FIG. 7 illustrates a perspective overview of said catheter bag holder attached to said user.

### DETAILED DESCRIPTION

Described herein is an improved catheter bag holder and method of use. The following description is presented to enable any person skilled in the art to make and use the invention as claimed and is provided in the context of the particular examples discussed below, variations of which will be readily apparent to those skilled in the art. In the interest of clarity, not all features of an actual implementation are described in this specification. It will be appreciated that in the development of any such actual implementation (as in any development project), design decisions must be made to achieve the designers' specific goals (e.g., compliance with system- and business-related constraints), and that these goals will vary from one implementation to another. It will also be appreciated that such development effort might be complex and time-consuming, but would nevertheless be a routine undertaking for those of ordinary skill in the field of the appropriate art having the benefit of this disclosure. Accordingly, the claims appended hereto are not intended to be limited by the disclosed embodiments, but are to be accorded their widest scope consistent with the principles and features disclosed herein.

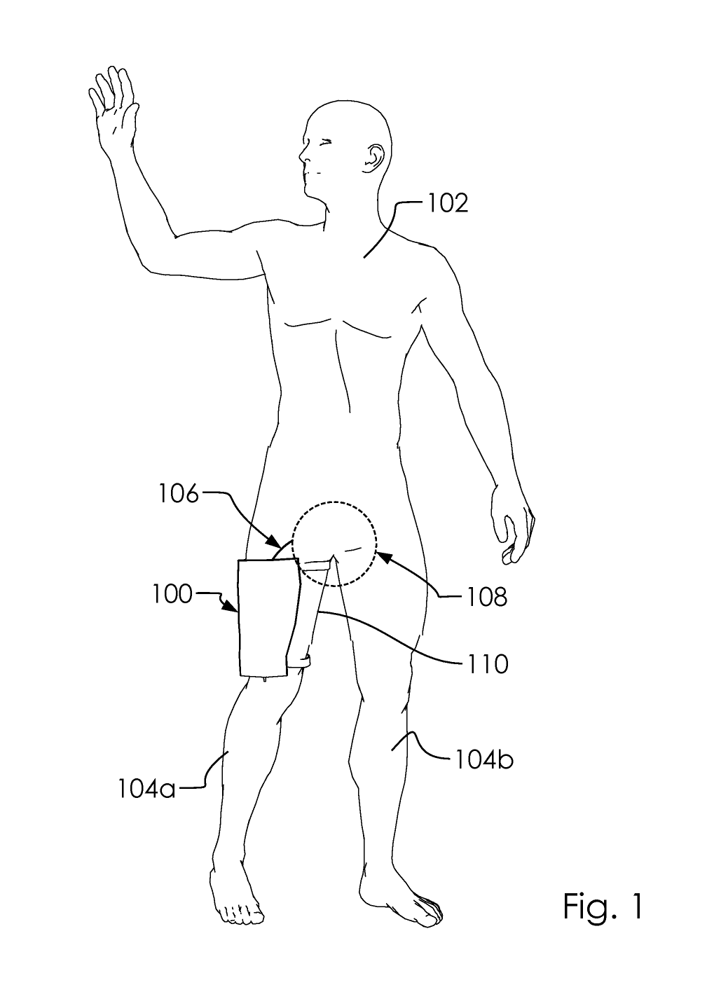

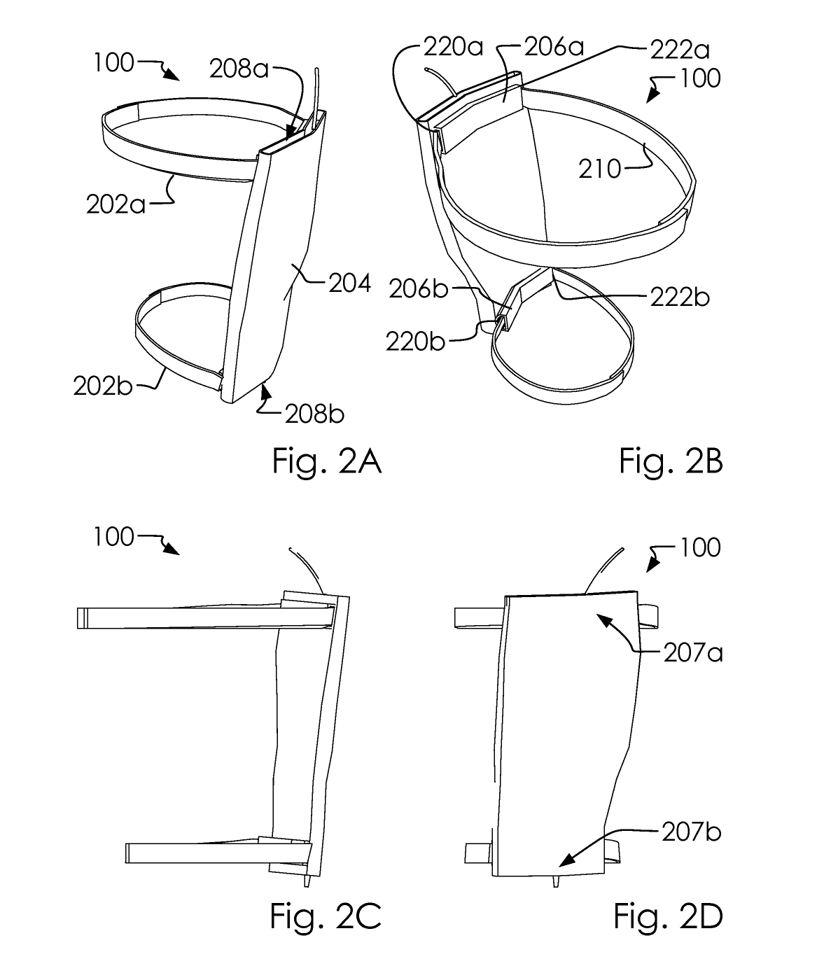

FIG. 1 illustrates a perspective overview of a catheter bag holder 100 on a user 102. In one embodiment, said catheter bag holder 100 can attach to a leg (such as a first leg 104a or a second leg 104b) of said user 102. As illustrated, said catheter bag holder 100 is attached to said first leg 104a. In one embodiment, said catheter bag holder 100 can hold a catheter system 106 (partially illustrated here). In one embodiment, said catheter bag holder 100 can allow an input tube 602 of said catheter system 106 to be ideally positioned for the collection bodily fluids (such as urine) at a groin area 108 of said user 102. In one embodiment, said catheter bag holder 100 can comprise a one or more straps which can comprise a first strap 202a and a second strap 202b (illustrated in FIG. 2 and following). In one embodiment, said one or more straps comprising a tubular shape. Thus, in one embodiment said catheter bag holder 100 can be adjustable so as to fit a wide range of body sizes for said user 102; wherein, said one or more straps comprise a circumference, and said circumference of said one or more straps are capable of adjusting to as to attach to said leg of said user with various circumferences. In one embodiment, said catheter bag holder 100 can attach around a thigh 110 of said leg of said user. In one embodiment, said one or more straps can comprise said circumference being adjustable in length so that said one or more straps are capable of attaching to said first leg 104a.

FIGS. 2A, 2B, 2C and 2D illustrate a perspective front and back view, an elevated side view and an elevated front view of said catheter bag holder 100. In one embodiment, said one or more straps can wrap around a portion of said first leg 104a and can be capable of holding said catheter bag holder 100 to said leg of said user by squeezing said leg of said user. In one embodiment, said one or more straps can comprise an elastic material such as a non-roll elastic strap; wherein, said one or more straps are designed not to roll on a leg of a user. An example of non-roll elastic has been included in the Invention Disclosure Statement. In one embodiment, said one or more straps can comprise an elastic interior portion wrapped by a non-elastic textile exterior 210; wherein, said non-elastic textile exterior 210 provides a lower friction surface for said one or more straps which can protect said first leg 104a.

In one embodiment, said one or more straps can attach to a pocket 204. In one embodiment, said pocket 204 can comprise a one or more sleeves (which can comprise a first sleeve 206a and a second sleeve 206b). In one embodiment, said pocket 204 can comprise a top portion 207a and a bottom portion 207b of said pocket 204. In one embodiment, a portion of said one or more straps can be threaded through said one or more sleeves and a portion of said one or more sleeves can be attached to said pocket 204. In one embodiment, said one or more sleeves can comprise a first side openings and second side openings; for examples, said first sleeve 206a can comprise a first side opening 220a and a second side opening 222a, and said second sleeve 206b can comprise a second side opening 220b and a second side opening 222b; wherein, said first side openings and said second side openings are substantially perpendicular to said top opening 208a and said bottom opening 208b. In one embodiment, said first strap 202a can slide through said first sleeve 206a and said second strap 202b can slide through said second sleeve 206b. In one embodiment, said one or more sleeves can be attached to said pocket 204 by sewing said one or more sleeves to said pocket 204. For example, in one embodiment, said first sleeve 206a can attach to said top portion 207a and said second sleeve 206b can attach to said bottom portion 207b. In one embodiment, said pocket 204 can hold a portion of said catheter system 106, as described below.

In one embodiment, said pocket 204 can comprise a top opening 208a and a bottom opening 208b. In one embodiment, said catheter system 106 can be put into said pocket 204 through said top opening 208a. In one embodiment, a weight of said catheter system 106 can be held inside of said pocket 204 and held to said leg of said user with said one or more straps.

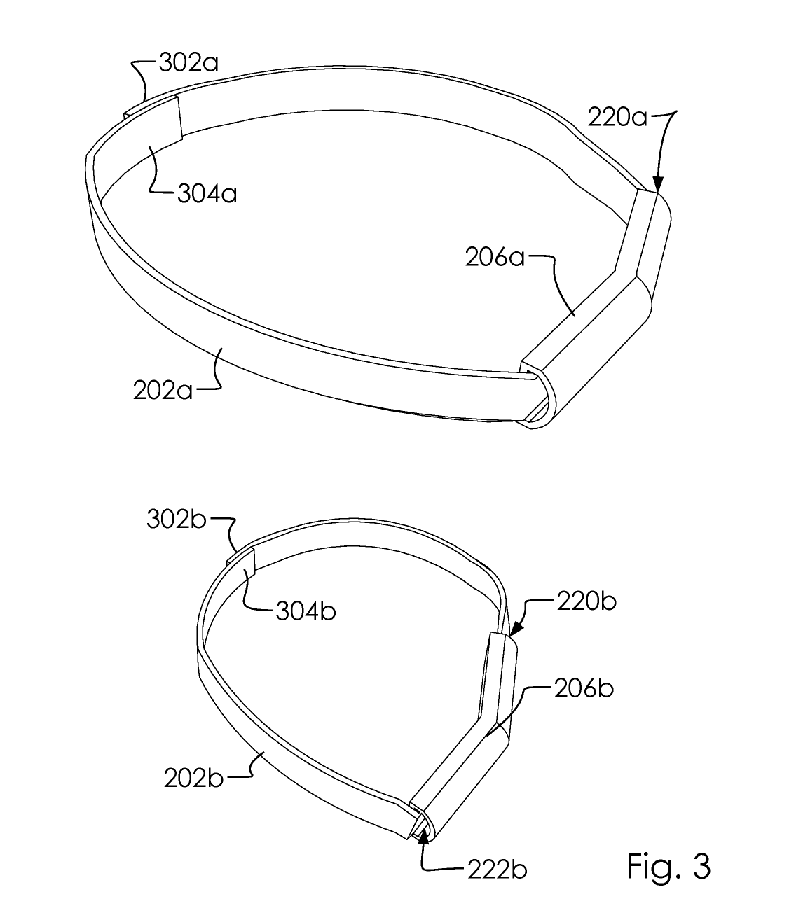

FIG. 3 illustrates a perspective overview of one embodiment of said one or more straps and said one or more sleeves. In one embodiment, said one or more straps can each comprise a first end and a second end. For example, in one embodiment, said first strap 202a can comprise a first end 302a and a second end 304a, and said second strap 202b can comprise a first end 302b and a second end 304b. In one embodiment, said first ends and said second ends of said one or more straps can attach to one another with a hook-and-loop fastener, as discussed below. For example, in one embodiment, said clasp or hook-and-loop fasteners can allow said one or more straps to be partially wrapped around said first leg 104a and attached to be held there;

further, to allow said one or more straps to be tightened and/or loosened as necessary. In one embodiment, where said one or more sleeves are attached to said pocket 204, said one or more straps can slide through said one or more sleeves and thereby adjust a centering of said one or more straps with respect to said pocket 204.

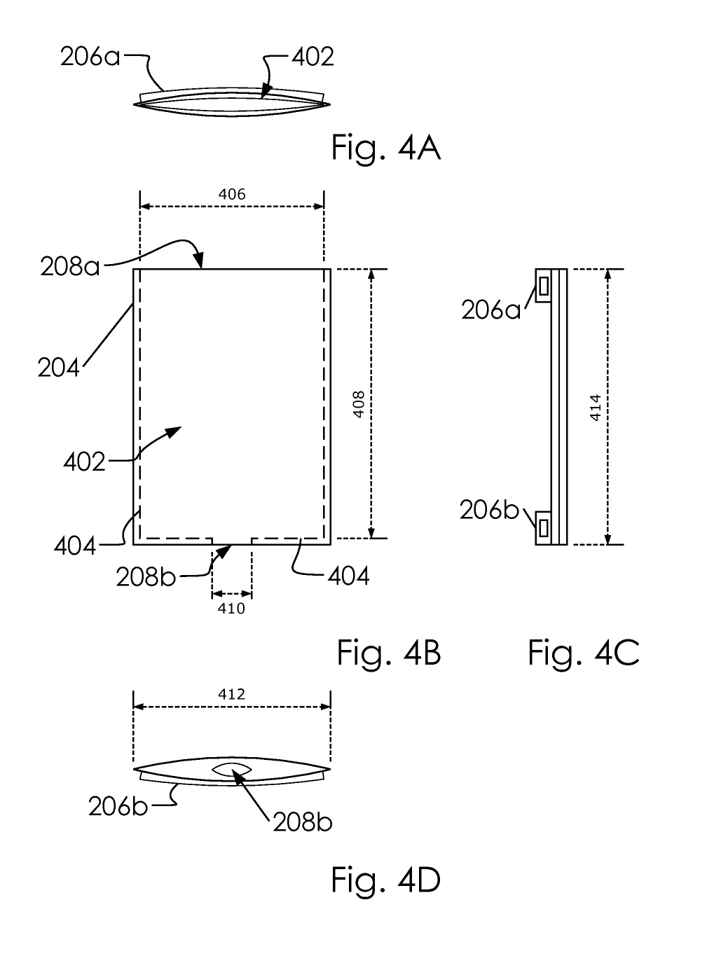

FIGS. 4A, 4B, 4C and 4D illustrate four elevated views of said pocket 204 with said one or more sleeves; including a top view, a front view, a side view and bottom view. In one embodiment, said pocket 204 can comprise a compartment 402. In one embodiment, said compartment 402 can comprise said top opening 208a and said bottom opening 208b. In one embodiment, said compartment 402 can hold a portion of said catheter system 106. FIG. 4B illustrates one embodiment of said compartment 402 by showing a seam 404 (as a dashed line) at the edges of said compartment 402 within said pocket 204; wherein, said seam 404 can define edgewise boundaries of said compartment 402. In one embodiment, said compartment 402 can be created by sewing said seam 404 into said pocket 204.

In one embodiment, said compartment 402 can comprise a pocket width 406 and a pocket height 408. In one embodiment, said top opening 208a can comprise an upper opening width at said top portion 207a of said pocket 204 equal to said pocket width 406 and a lower opening width 410 at said bottom portion 207b of said pocket 204. In one embodiment, said pocket width 406 can be larger than said lower opening width 410. In one embodiment, said pocket 204 can have a bag width 412 and a bag height 414. In one embodiment, said catheter bag holder 100 can accommodate different capacity sizes of said catheter system 106 by altering the size of said compartment 402 and the weight carrying capacity of said one or more straps. It will be understood by one of ordinary skill in the art that such size and capacity changes can be made without substantial guesswork.

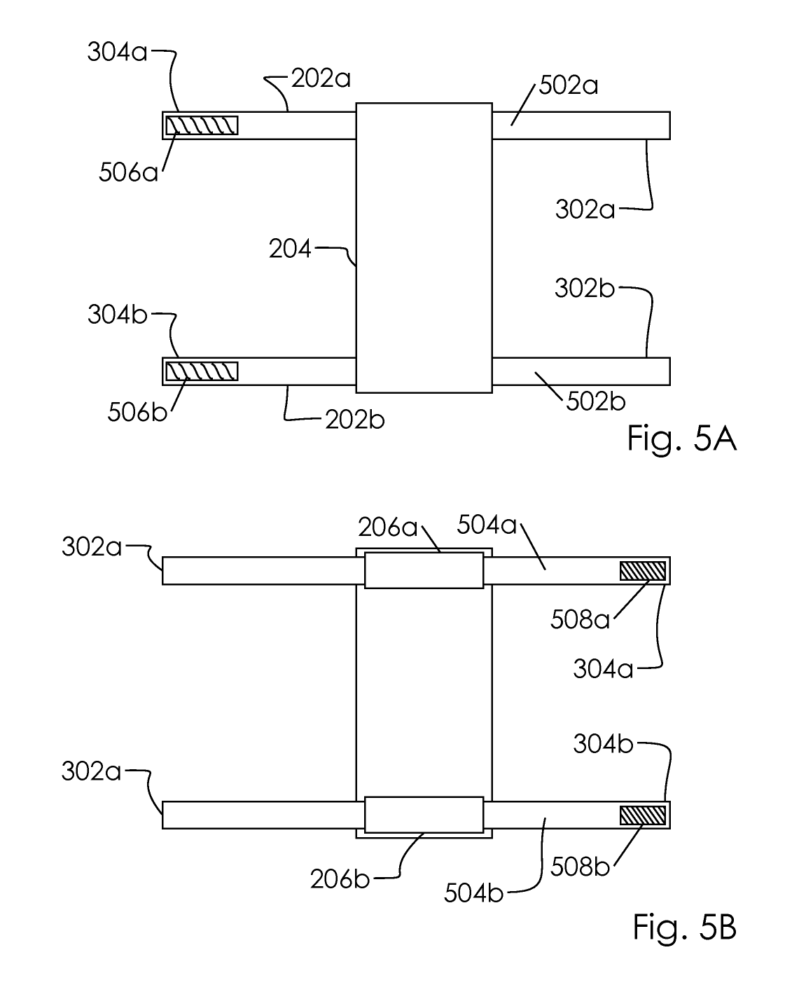

FIGS. 5A and 5B illustrate an elevated front view and an elevated rear view of said catheter bag holder 100. In one embodiment, said one or more straps can each comprise said a one or more hook-and-loop fasteners. For example, in one embodiment, said one or more straps can each comprise a first side and a second side; wherein, said hook-and-loop fasteners can attach partially to said first side and partially to said second side of said one or more straps; and, said one or more straps can wrap around said leg of said user and attach to one another with said first side and said second side releaseably attaching to one another. For example, in one embodiment, said first strap 202a can comprise a first side 502a having a first portion 506a of said hook-and-loop fasteners and a second side 504a can comprise a second portion 508a of said hook-and-loop fasteners; likewise, said second strap 202b can comprise a first side 502b having a first portion 506b of said hook-and-loop fasteners and a second side 504b can comprise a second portion 508b of aid hook-and-loop fasteners.

FIG. 5C illustrates said catheter bag holder 100 with a one or more buckles. In one embodiment, said one or more buckles can comprise a first buckle 510a at said second end 304a of said first strap 202a, and a second buckle 510b at said second end 304b of said second strap 202b. In one embodiment, said one or more buckles can attach to said first ends of said one or more straps; for example, said first end 302a can attach to said second end 304a with said first buckle 510a, and said first end 302b can attach to said second end 304b with said second buckle 510b.

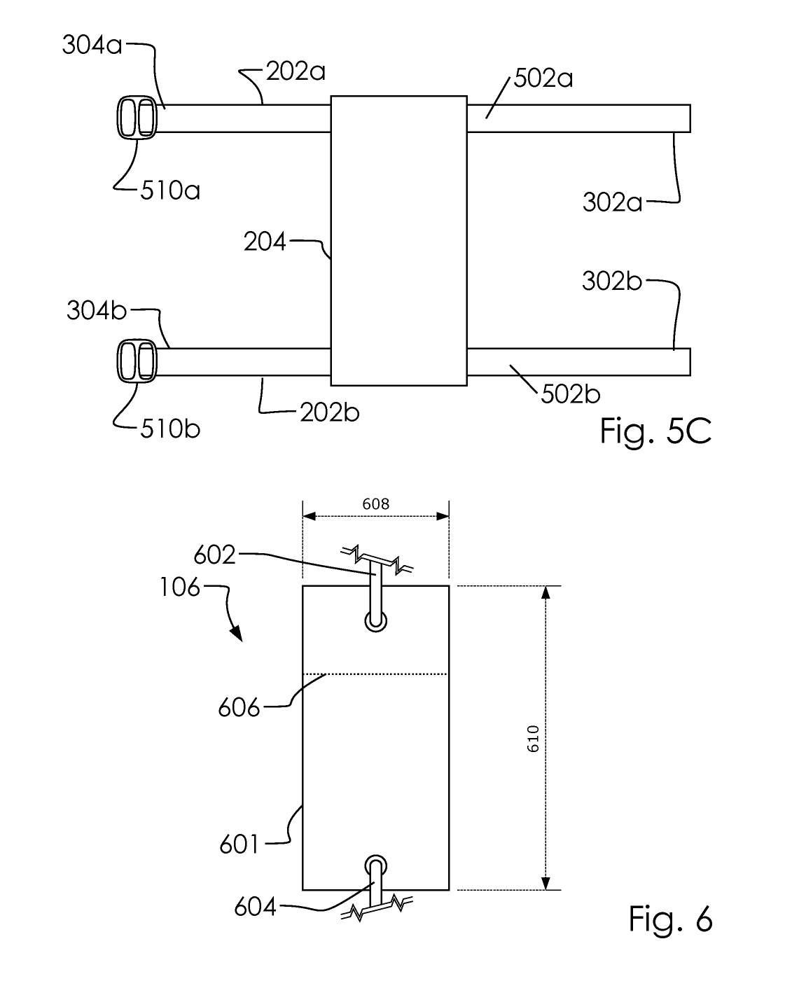

FIG. 6 illustrates an elevated front view of said catheter system 106. In one embodiment, said catheter system 106 can comprise a fluid bag 601 being sealed but-for said input tube 602 and an output tube 604. In one embodiment, said fluid bag 601 can receive a fluid 606 through said input tube 602 and drain said fluid 606 through said output tube 604. In one embodiment, a catheter can be attached to said input tube 602 as is known in the art. In one embodiment, said catheter bag holder 100 can allow said input tube 602 to pass through said top opening 208a and said output tube 604 to pass through said bottom opening 208b. In one embodiment, said fluid bag 601 can comprise a bag width 608 and a bag height 610. In one embodiment, said bag width 608 can be smaller than said pocket width 406 of said compartment 402 in said catheter bag holder 100. In one embodiment, said bag height 610 can be smaller than said pocket height 408 of said compartment 402, but it need not be so in order for said catheter bag holder 100 to function properly.

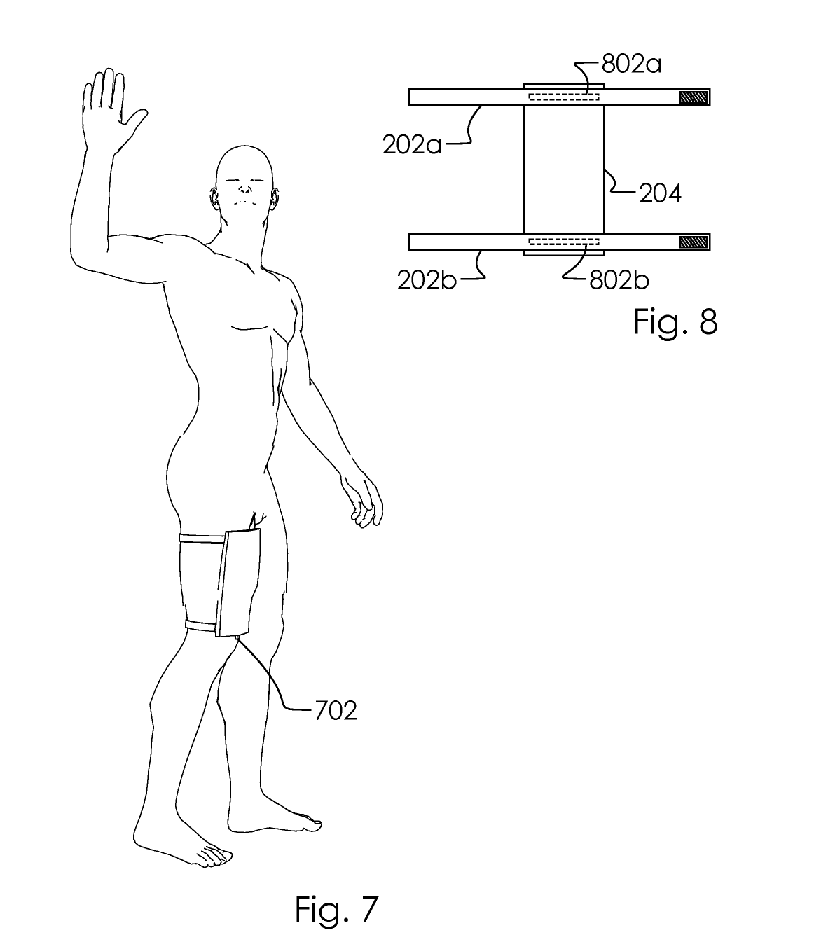

FIG. 7 illustrates a perspective overview of said catheter bag holder 100 attached to said user 102. In one embodiment, said catheter bag holder 100 can more comfortably hold said catheter system 106 on said first leg 104a than any known alternate systems. In one embodiment, said bottom opening 208b can allow said output tube 604 to drain from within said catheter bag holder 100 at the will of said user 102 by releasing and engaging a valve 702 on said output tube 604. Further, in one embodiment, said catheter bag holder 100 can be hidden below a garment of said user 102. In one embodiment, said garment can comprise short pants. Thus, said user 102 can use said catheter bag holder 100 in a very comfortable manner as opposed to dealing with said catheter system 106 in a conventional manner.

FIG. 8 illustrates an elevated backside view said catheter bag holder 100 without said one or more straps. In one embodiment, said one or more straps can be attached directly to said pocket 204 with a stitch. In one embodiment, a first stitch 802a can attach said first strap 202a to said pocket 204 and a second stitch 802b can attach said second strap 202b to said pocket 204.

Various changes in the details of the illustrated operational methods are possible without departing from the scope of the following claims. Some embodiments may combine the activities described herein as being separate steps. Similarly, one or more of the described steps may be omitted, depending upon the specific operational environment the method is being implemented in. It is to be understood that the above description is intended to be illustrative, and not restrictive. For example, the above-described embodiments may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.”

1. A catheter bag holder comprising:
a catheter bag holder comprising a pocket having a top opening, a bottom opening, a pocket width and a pocket height;
a one or more straps comprising a first strap and a second strap;
said pocket having a top portion and a bottom portion;
said top opening is in said top portion and said bottom opening is in said bottom portion;
said one or more straps comprise an elastic material;
said top opening having a top opening width and said bottom opening having a bottom opening width;
said pocket width is greater than or equal to a bag width of a fluid bag;
said bottom opening width of said pocket is smaller than said bag width;
said first strap is attached to said top portion of said pocket;
said second strap is attached to said bottom portion of said pocket;
said pocket receiving a input tube through said top opening a one or more sleeves comprise a first sleeve and a second sleeve;
said first sleeve is attached to said bag at said top portion;
said second sleeve is attached to said bag at said bottom portion;
said one or more sleeves comprising a tubular shape;
said one or more straps each comprise a first end and a second end;
said first strap slides through said first sleeve;
said second strap slides through said second sleeve;
a first end of said first strap attaches to a second end of said first strap;
a first end of said second strap attaches to a second end of said second strap;
said first end and said second end of said one or more straps attach to one another with a one or more hook-and-loop fasteners;
said one or more hook-and-loop fasteners comprise a first portion and a second portion on each of said one or more straps;
at said first strap
a first portion of said one or more hook-and-loop fasteners at said first end of said first strap releaseably attaches
to a second portion of said one or more hook-and-loop fasteners at said second end of said first strap; and
at said second strap
a first portion of said one or more hook-and-loop fasteners at said first end of said second strap releaseably attaches
to a second portion of said one or more hook-and-loop fasteners at said second end of said second strap.

2. A catheter bag holder comprising:
a catheter bag holder comprising a pocket having a top opening, a bottom opening, a pocket width and a pocket height;
a one or more straps comprising a first strap and a second strap;
said pocket having a top portion and a bottom portion;
said top opening is in said top portion and said bottom opening is in said bottom portion;
said one or more straps comprise an elastic material;
said top opening having a top opening width and said bottom opening having a bottom opening width;
said pocket width is greater than or equal to a bag width of a fluid bag;
said bottom opening width of said pocket is smaller than said bag width;
said first strap is attached to said top portion of said pocket;
said second strap is attached to said bottom portion of said pocket; and
said pocket receiving a input tube through said top opening.

3. The catheter bag holder of claim 2 further comprising a one or more sleeves; wherein, said one or more sleeves comprise a first sleeve and a second sleeve;
said first sleeve is attached to said bag at said top portion;
said second sleeve is attached to said bag at said bottom portion;
said one or more sleeves comprising a tubular shape;
said one or more straps each comprise a first end and a second end;
said first strap slides through said first sleeve;
said second strap slides through said second sleeve;
a first end of said first strap attaches to a second end of said first strap; and
a first end of said second strap attaches to a second end of said second strap.

4. The catheter bag holder of claim 3 wherein, said first end and said second end of said one or more straps attach to one another with a one or more hook-and-loop fasteners;
said one or more hook-and-loop fasteners comprise a first portion and a second portion on each of said one or more straps;
at said first strap
a first portion of said one or more hook-and-loop fasteners at said first end of said first strap releaseably attaches
to a second portion of said one or more hook-and-loop fasteners at said second end of said first strap; and
at said second strap
a first portion of said one or more hook-and-loop fasteners at said first end of said second strap releaseably attaches
to a second portion of said one or more hook-and-loop fasteners at said second end of said second strap.

5. The catheter bag holder of claim 3 wherein,
said first end and said second end of said one or more straps attach to one another with a one or more buckles.

6. The catheter bag holder of claim 5 wherein,
said one or more buckles comprise a first buckle and a second buckle;
said first buckle is attached at said second end of said first strap;
said first buckle releaseably attaches to said second end of said first strap;
said second buckle is attached at said second end of said second strap; and
said second buckle releaseably attaches to said second end of said second strap.

7. The catheter bag holder of claim 2 wherein,
said one or more straps each comprise a first end and a second end; and
said first end and said second end of said one or more straps attach to said bag.

8. The catheter bag holder of claim 2 wherein,
said one or more straps each form a loop; and
said loop in said one or more straps comprises a circumference being adjustable in length so that said one or more straps are capable of attaching to a leg of a user.

9. The catheter bag holder of claim 2 wherein,
said one or more straps comprise an elastic interior portion wrapped by a non-elastic textile exterior.

10. The catheter bag holder of claim 2 wherein,
said one or more straps each comprise a first opening and a second opening; and
said first opening and said second opening of said one or more straps being substantially perpendicular to said top opening and said bottom opening of said pocket of said catheter bag holder.

11. The catheter bag holder of claim 2 wherein,
said pocket comprises a compartment within said pocket and between said top opening and said bottom opening; and
said compartment can be formed by sewing a seam at one or more edges of said pocket and leaving said top opening and said bottom opening unbound.

12. The catheter bag holder of claim 2 wherein,
said pocket comprising a compartment;
said compartment being adapted to holding a fluid bag having a input tube at a top portion of said fluid bag and a output tube at a bottom portion of said fluid bag;
said input tube being attached to a catheter which is attached to a user of said catheter bag holder and collecting a fluid from said user; and
said output tube comprising a valve capable of selectively relieving said fluid from said fluid bag.

13. A method of using a catheter bag holder comprising:
attaching a catheter bag holder to a first leg of a user of said catheter bag holder by wrapping a one or more straps around a thigh of said first leg of said user,
attaching said one or more straps to a pocket of said catheter bag holder,
attaching a catheter to a groin area of said user for collection of bodily fluids from said user,
attaching said catheter to a input tube of a fluid bag,
inserting said fluid bag into said pocket of said catheter bag holder through a top opening of said catheter bag holder, and
running a output tube from said fluid bag through a bottom opening of said pocket;
and wherein,
said catheter bag holder comprises said pocket having a pocket width and a pocket height,
said one or more straps comprising a first strap and a second strap,
said pocket comprises a top portion and a bottom portion,
said top opening is in said top portion and said bottom opening is in said bottom portion,
said one or more straps comprise an elastic material,
said top opening having a top opening width and said bottom opening having a bottom opening width,
said pocket width is greater than or equal to a bag width of said fluid bag,
said bottom opening width of said pocket is smaller than said bag width,
said first strap is attached to said top portion of said pocket,
said second strap is attached to said bottom portion of said pocket, and
said pocket receiving a input tube through said top opening.

14. The method of using a catheter bag holder of claim 13 wherein, attaching said one or more straps to a pocket of said catheter bag holder comprises:
attaching a one or more sleeves to said pocket,
sliding a portion of said one or more straps through said one or more sleeves, and
securing a first end to a second end of said one or more straps; and wherein,
said one or more sleeves comprise a first sleeve and a second sleeve,
said first sleeve is attached to said bag at said top portion,
said second sleeve is attached to said bag at said bottom portion,
said one or more sleeves comprising a tubular shape,
said one or more straps each comprise said first end and said second end,
said first strap slides through said first sleeve,
said second strap slides through said second sleeve,
a first end of said first strap attaches to a second end of said first strap, and
a first end of said second strap attaches to a second end of said second strap.

15. The method of using a catheter bag holder of claim 13 wherein, wrapping a one or more straps around a thigh of said first leg of said user comprises:
forming a loop with each of said one or more straps, and
wrapping and tightening said loops of said one or more straps around said thigh of said user; and wherein,
said loop in said one or more straps comprises a circumference, and
said circumference of said loops are adjustable in length so that said one or more straps are capable of attaching to a leg of a user.

16. The method of using a catheter bag holder of claim 13 further comprising:
emptying said fluid bag of said bodily fluids by releasing a valve on said output tube and
allowing said bodily fluids to drain from said fluid bag through said bottom opening of said pocket.
